(19) United States
(12) Patent Application Publication
(10) Pub. No.: US 2025/0381335 A1
(43) Pub. Date: Dec. 18, 2025

(54) PHYSIOLOGIC CARDIOVASCULAR PH BALANCED COUNTER CURRENT ELECTROLYTE TRANSFER AND FLUID REMOVAL SYSTEM

(71) Applicant: NephraTech LLC, Austin, TX (US)

(72) Inventors: Richard Blake DENISON, Austin, TX (US); Mary Claire MC Garry, Austin, TX (US)

(21) Appl. No.: 19/302,738

(22) Filed: Aug. 18, 2025

Related U.S. Application Data
(63) Continuation of application No. 17/048,855, filed on Oct. 19, 2020, now Pat. No. 12,415,025.
(60) Provisional application No. 62/659,951, filed on Apr. 19, 2018.

(51) Int. Cl. A61M 1/36; A61M 1/16; A61M 1/34

(52) U.S. Cl. CPC A61M 1/3666; A61M 1/1698; A61M 1/3403; A61M 1/3609; A61M 1/3623; A61M 2230/208

(57) ABSTRACT

Certain embodiments of the invention are directed to methods of adjusting the concentration of one or more electrolytes in a patient's blood using a counter current electrolyte solution.

### CROSS-REFERENCE TO RELATED APPLICATIONS

This application claims the benefit of priority to U.S. Provisional Patent Application Ser. No. 62/659,951, filed Apr. 19, 2018, hereby incorporated by reference in its entirety.

### BACKGROUND OF THE INVENTION

### A. Field of the Invention

The invention generally concerns a system or device for adjusting the concentration of at least one electrolyte in a fluid, such as blood, and methods related to the same.

### B. Description of Related Art

In order for the cardiac surgeon to repair cardiac defects, the majority of the operations require the use of cardiopulmonary bypass. Cardiopulmonary bypass includes a heart/lung machine, with various pumps, an artificial lung or oxygenator, a heater/cooler for temperature control and an artificial kidney or hemofilter. None of these features of cardiopulmonary bypass is actually physiological in comparison to the patient's physiological process under normal circumstances. However, the clinical practitioners or perfusionist is in charge of monitoring and maintaining physiological conditions of the patient's circulation, ventilation, oxygenation, anticoagulation, fluid and electrolyte balance throughout the entire bypass surgery. Although, there have been vast improvements in the heart/lung machine, as well as the disposables over the past half century, the process of fluid and electrolyte balance has not changed much, if at all.

The accepted crystalloid fluids, 0.9% NaCl and Plasmalyte, which are used during cardiopulmonary bypass have been unchanged for the last few decades. If these crystalloids are examined closely, it is found that these solutions have a low pH and the sodium and chloride concentration (0.9% NaCl) are not physiological. But, the industry wide accepted practice to attempt to manipulate the electrolytes begins with the perfusionist selecting a crystalloid solution that lacks the electrolyte value they are trying to change. For instance, if a patient's elevated potassium level is to be reduced, the perfusionist will most likely select 0.9% NaCl as their crystalloid, which is potassium free. The process begins by adding 0.9% NaCl into the patient's bypass circuit reservoir then removing it via a hemofilter. By adding this potassium free solution into the circuit and filtering with ultrafiltration, the potassium level in the patient's blood will eventually be reduced. This accepted process is inefficient, as well as leaving the patient's blood in a more acidotic state. To bring the pH back into a physiological range, it is necessary to treat the induced acidosis by administering sodium bicarbonate, which in turn causes the sodium level of the patient's blood to become elevated outside of the normal sodium physiological range. As can be seen, to remedy one electrolyte, another electrolyte becomes out of range. Even in routine cases, many liters of a crystalloid may be added to the patient's bypass circuit to “wash” the ultrafiltration filtrate in an attempt to maintain the patient in as close to a physiological state as possible. This antiquated, yet singular option for restoring the electrolytes, causes a host of other imbalances. The patient can all but be guaranteed metabolic acidosis, hypocalcemia, hyperchloremia, and/or hypernatremia.

There is a need for additional devices, systems, and methods for restoring or maintaining electrolyte balance of blood during medical procedures.

### SUMMARY OF THE INVENTION

The traditional method of achieving electrolyte balance while on cardiopulmonary bypass is slow, inefficient, unreliable and has not changed for decades. The device, system, and/or methods described herein can be employed during cardiopulmonary bypass to reduce or balance electrolytes in a quick and safe manner. The conventional hemofilter is modified to facilitate the transfer of electrolytes, as well as facilitating fluid removal from the circuit. The PH balanced electrolyte solutions are customized to maintain the physiological conditions of the patients, for example, during heart surgery.

Embodiments of devices or systems described herein can be incorporated into or used with a cardiopulmonary bypass machine so that electrolyte balance can be regulated in a faster and more efficient manner, providing a solution to the electrolyte and pH problems associated with current methods that use crystalloids. In particular, the device and methods described herein safely and quickly change electrolytes within the bypass/patient circulation.

In certain embodiments the devices, systems, or methods described herein is capable of restoring banked blood, regardless of its age or decomposition. By way of example, the inventors have designed a system and related process to quickly and efficient maintain electrolyte balance and pH of blood, as well as restore such in non-circulating blood, which results in a more physiologically acceptable blood product having appropriate electrolyte balance and pH.

The features of the devices and methods described herein allow for the safe transfer of electrolytes during counter current flow. Dual pumps can be controlled together or individually depending on mode selection. The term “mode” is used to designate an operating process that produces a desired result. Modes include, but are not limited to (1) “zero balanced” electrolyte transfer mode, (2) fluid removal mode, (3) a combination of both electrolyte transfer and fluid removal mode, and (4) physiological restoration mode. The various modes provide a user with the appropriate functionality to maintain blood or a patient at or about a physiologic condition. The banked blood mode allows for the restoration of banked blood to an approximate normal physiologic state. Devices described herein can be portable. The portability of the device allows its use in a variety of locations, such as areas of a medical facility, e.g., operating rooms (OR), intensive care units (ICU), emergency rooms (ER) or blood banks.

Certain embodiments of the invention are directed to methods of adjusting the concentration of one or more electrolytes in a patient's blood, the method comprising: (a) providing an extracorporeal blood circuit comprising the following components in fluid communication with a patient's circulatory system: blood flow tubing, an oxygenator, a blood pump, and a hemofilter, wherein the hemofilter comprises a first filter chamber and a second filter chamber separated by a semi-permeable barrier; (b) flowing the patient's blood through the first filter chamber; (c) adjusting the concentration of at least one electrolyte in the patient's blood by flowing a first wash solution through the second filter chamber in a direction of flow opposite to the direction which the patient's blood is flowing through the first filter chamber. In certain aspects the at least one electrolyte in the patient's blood comprises at least one of hydrogen ion, sodium, potassium, calcium, chloride, glucose, bicarbonate, and lactate. In certain aspects at least one electrolyte is hydrogen ions and 1, 2, 3, 4, 5, 6, or 7 of sodium, potassium, calcium, chloride, glucose, bicarbonate, and lactate. In certain aspects at least one electrolyte is sodium and 1, 2, 3, 4, 5, 6, or 7 of hydrogen ions, potassium, calcium, chloride, glucose, bicarbonate, and lactate. In a further aspect at least one electrolyte is potassium and 1, 2, 3, 4, 5, 6, or 7 of hydrogen ions, sodium, calcium, chloride, glucose, bicarbonate, and lactate. In a further aspect at least one electrolyte is calcium and 1, 2, 3, 4, 5, 6, or 7 of hydrogen ions, sodium, potassium, chloride, glucose, bicarbonate, and lactate. In a further aspect at least one electrolyte is chloride and 1, 2, 3, 4, 5, 6, or 7 of hydrogen ions, sodium, potassium, calcium, glucose, bicarbonate, and lactate. In still a further aspect at least one electrolyte is glucose and 1, 2, 3, 4, 5, 6, or 7 of hydrogen ions, sodium, potassium, calcium, chloride, bicarbonate, and lactate. In a certain aspect at least one electrolyte is bicarbonate and 1, 2, 3, 4, 5, 6, or 7 of hydrogen ions, sodium, potassium, calcium, chloride, glucose, and lactate. In a certain aspect at least one electrolyte is lactate and 1, 2, 3, 4, 5, 6, or 7 of hydrogen ions, sodium, potassium, calcium, chloride, glucose, and bicarbonate. Step (c) can be performed for a time sufficient to cause the concentration of the at least one electrolyte in the patient's blood to adjust from a concentration outside a desired range to a concentration within the desired range. In certain aspects the desired range for hydrogen ion is pH 7.35 to 7.45, the desired range for sodium is 135 to 145 mM, the desired range for potassium is 3.5 to 4.5 mM, the desired range for calcium is 0.8 to 1.2 mM, the desired range for chloride is 95 to 115 mM, the desired range for glucose is 80 to 120 mg/dL, the desired range for bicarbonate is 22 to 28 mM, and the desired range for lactate is 0 to 2 mM. When performing step (c) the concentration of the at least one electrolyte is adjusted to a concentration within the desired range within 4, 6, 8, 10, 15, 20 minutes, including all values there between (e.g., 4, 6, 8 to 10, 15, 20 minutes), of initiating flow of the first wash solution through the second filter chamber. In a particular aspect the concentration of the at least one electrolyte is adjusted to a concentration within the desired range within 6 minutes of initiating flow of the first wash solution through the second filter chamber. In certain aspects performance of step (c) causes the concentration of the at least one electrolyte to adjust by at least 5, 10, 15, 20, 25, 30%, including all values there between (e.g., 5, 10, 15 to 20, 25, 30%, 5 to 15%, 10 to 15%, 10 to 20%, 15 to 30%), within 3 minutes. In particular aspects the concentration of the at least one electrolyte to adjust is changed by at least 15% within 3 minutes.

In certain embodiments the concentration of an electrolyte in the wash solution can be adjusted to effect a desired change in the electrolyte in the blood, e.g., if a blood electrolyte is elevated the wash solution can have a lower concentration of that electrolyte relative to the blood, alternatively if the blood electrolyte is low the was solution can have a higher concentration of that electrolyte relative to the blood. In certain aspects the first wash solution either does not comprise the at least one electrolyte (a target electrolyte) or comprises the at least one electrolyte at a concentration below the concentration of the at least one electrolyte in the blood before step (c) is performed. In a further aspect, the first wash solution comprises the at least one electrolyte at a concentration above the concentration of the at least one electrolyte in the blood before step (c) is performed. In a particular embodiments the first wash solution can have a pH of 7.35 to 7.45 and comprises 135-145 mmol/L of sodium, 22-28 mmol/L bicarbonate, 80-100 mmol/L glucose, 0.8-1.0 mmol/L calcium, 0 mmol/L potassium in one wash solution and 3.5 mmol/L potassium in another. In certain aspects the volume of the first wash solution flowed through the second filter chamber in step (c) is less than 200, 300, 400, 500, 600, 700, 800, 900, or 1000 mL, including all values there between, e.g., 200 to 1000 mL, 500 to 700 mL. In a particular aspect the volume of the first wash solution flowed through the second filter chamber in step (c) is less than 600 mL. The rate of flow of the first wash solution through the second filter chamber can be from 20, 30, 40, 50 to 60, 70, 80, 90 100 ml/min, including all values and ranges there between. The rate of flow of the patient's blood through the first filter chamber can be from 20, 30, 40, 50, 60, 70, 80, 90, 100 to 120, 140, 160, 180, 200 ml/min, including any value and range there between.

The method can further comprise stopping the flow of wash solution through the second filter chamber when the concentration of the at least one electrolyte in the patient's blood is within a desired range. In certain aspects the method can further comprise flowing a second wash solution through the second filter chamber after the concentration of the at least one electrolyte in the patient's blood is adjusted to be within a desired range, wherein the second wash solution comprises the at least one electrolyte at a concentration within the desired range.

The method can further comprise monitoring the concentration of the at least one electrolyte in the patient's blood. In certain aspects the concentration of sodium in the patient's blood remains below 140 mM or does not increase by more than 5% while the method is being performed. In a further aspect, performance of the method does not cause a decrease in the hematocrit of the patient's blood of more than 1, 2, 3, 4, 5, to 6%, including all values there between, e.g., 1 to 6%, 2 to 4%, etc. In particular aspects, performance of the method does not cause a decrease in the hematocrit of the patient's blood of more than 3%.

In certain aspects, step (c) further comprises: (i) flowing the wash solution from a wash solution reservoir through wash solution tubing coupled to a wash solution inlet port, the wash solution inlet port being in fluid communication with the second filter chamber; and (ii) flowing wash solution effluent from an effluent outlet port in fluid communication with the second filter chamber and through effluent tubing coupled to the effluent output port.

In certain embodiments the blood flow tubing is coupled to a blood input port and a blood outlet port, the blood input port and the blood output port being in fluid communication with the first filter chamber. An extracorporeal blood circuit can be coupled to a heat exchange unit. In certain aspects the extracorporeal blood circuit is comprised in a cardiopulmonary bypass system. In a further aspect the extracorporeal blood circuit is comprised in an extracorporeal membrane oxygenation system. In certain embodiments the patient is undergoing cardiac surgery. The patient can be of any age. In certain aspects the patient is 1 to 12 months old, 1, 10, 20, 30, 40 to 50, 60, 70, 80, 90 years old, including all ages there between.

In certain aspects the concentration of the at least one electrolyte is adjusted without adding fluid to a reservoir of the patient's blood. In a further aspect the concentration of the at least one electrolyte is adjusted without a transient or permanent net influx of fluid into the patient's blood. Traditional ultrafiltration methods add the wash solution into the patient circuit reservoir making direct contact with the patient's blood circulation. In certain aspects of the current method, the wash solution comes in contact with a counter current semipermeable membrane inside the hemoconcentrator, pulling fluid across the membrane, providing a more efficient method. In still a further aspect the concentration of the at least one electrolyte is adjusted without a transient or permanent net efflux of fluid from the patient's blood. In certain aspects the volume of the patient's blood in the extracorporeal circuit remains constant throughout the performance of the method. In other aspects the volume of the patient's blood in the extracorporeal circuit does not vary by more than 0.5, 1, 2, 3, 4, 5% throughout performance of the method. In a particular aspect the volume of the patient's blood in the extracorporeal circuit does not vary by more than 1% throughout performance of the method. In certain aspects the concentration of the at least one electrolyte is adjusted without addition of fluid to a patient's blood other than fluid that flows across the semipermeable barrier in the filter. In certain aspects the concentration of the at least one electrolyte is adjusted without withdrawing fluid from the patient's blood other than fluid that flows across the semipermeable barrier in the filter.

In certain aspects the second filter chamber is in fluid communication with a wash solution input pump controlling the rate of flow of wash solution into the second filter chamber and an effluent pump controlling the rate of flow of effluent out of the second filter chamber. The method can further comprise adjusting the rates of the wash solution input pump and the effluent pump to cause a lower pressure in the second filter chamber than the pressure in the first filter chamber and a net efflux of fluid from the patient's blood. The method can further comprise measuring the pressure in the second filter chamber. In certain aspects the pressure in the second filter chamber is 0 to −300 mmHg relative to the first filter chamber. The method can include adjusting the rate of the wash solution input pump and/or the effluent outlet pump based on the pressure measurement or pressure differential between the first filter chamber and the second filter chamber. In certain aspects the method includes adjusting the rate of the wash solution input pump and/or the effluent pump comprises stopping the wash solution input pump and/or the effluent pump if the pressure in the second filter chamber is higher than the pressure in the first filter chamber. In certain aspects the second filter chamber can be in fluid communication with a plurality of wash solutions, 2, 3, 4, 5, 6, 7, 8, 9, 10 or more wash solutions, including all values and ranges there between.

In certain aspects the wash solution input pump and the effluent outlet pump are electrically coupled to (i) a sensor that senses the concentration of the at least one electrolyte in the patient's blood and (ii) a processor that receives electrolyte concentration data from the sensor and controls the rate of flow of wash solution through the second filter chamber by adjusting the rates of the wash solution input pump and the effluent pump based on the electrolyte concentration data.

Certain embodiments are directed to methods of preparing stored blood for injection into a patient comprising. The methods can include (a) providing a filter in fluid communication with a reservoir containing the stored blood, wherein the filter comprises a first filter chamber and a second filter chamber separated by a semipermeable barrier; (b) flowing the stored blood through the first filter chamber; (c) adjusting the concentration of at Least one electrolyte in the stored banked blood by flowing a wash solution through the second filter chamber in a direction of flow opposite to the direction that the patient's blood is flowing through the first filter chamber.

Certain embodiments are directed to a system for adjusting the concentration of at least one electrolyte in a patient's blood. The system can include (a) blood flow tubing in fluid communication with the patient's circulatory system; (b) an oxygenator in fluid communication with the blood flow tubing; (c) a blood pump that causes blood to flow within the blood flow tubing; (d) a filter comprising a first filter chamber and a second filter chamber separated by a semipermeable barrier, wherein the first filter chamber is in fluid communication with the blood flow tubing; (e) a wash solution reservoir in fluid communication with the second filter chamber; and (f) a wash solution input pump in fluid communication with the wash solution reservoir that causes the wash solution to flow within the second filter chamber in a direction opposite the direction of blood flow. In certain aspects the wash solution input pump is upstream from and in fluid communication with a wash solution inlet port, the wash solution inlet port being in fluid communication with the second filter chamber. The system can further include an effluent pump downstream from and in fluid communication with an effluent outlet port, the effluent outlet port being in fluid communication with the second filter chamber. In certain aspects the system includes a pump speed controller that can adjust the speed of the wash solution input pump and/or the effluent pump. The system can also include a display that indicates the rate of the wash solution input pump and/or the effluent pump. In certain aspects the wash solution input pump, the effluent pump, the pump speed controller, and the display are comprised in a single, portable console. The system can also include a sensor that can detect the concentration of at least one electrolyte in the patient's blood. The sensor can be configured to communicate electrolyte concentration data to a processor. The processor can cause a change in speed of the wash solution input pump and/or the effluent pump based on the electrolyte concentration data. The system can also include a pressure sensor in fluid communication with the second filter chamber. A safety shut-off mechanism can be included that can stop the wash solution pump and/or the effluent pump if the pressure in the second filter chamber is outside a predetermined range. The wash solution reservoir can contain a wash solution having 133145 mmol/L sodium, a pH of 7.35-7.45, 22-28 mmol/L bicarbonate, 95-115 mmol/L chloride.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa.

The term “crystalloid” refers to small molecules (usually less than 10 Å) such as salts, sugars, and buffers.

The use of the word “a” or “an” when used in conjunction with the term “comprising” in the claims and/or the specification may mean “one,” but it is also consistent with the meaning of “one or more,” “at least one,” and “one or more than one.”

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The term “substantially” and its variations are defined to include ranges within 10%, within 5%, within 1%, or within 0.5%.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods of making and using the same of the present

invention can “comprise,” “consist essentially of,” or “consist of” particular ingredients, components, blends, method steps, etc., disclosed throughout the specification.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE SEVERAL VIEWS OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. This drawing illustrates one embodiment of a device as described herein.

FIG. 2. This drawing illustrates a second embodiment of a device as described herein, this device is configured for blood restoration.

### DETAILED DESCRIPTION OF THE INVENTION

The device, system, and methods described herein can be used to maintain the physiologic state of a patient through the entire electrolyte spectrum. It began with the quest to create a crystalloid solution that would maintain the appropriate electrolyte ranges while lowering targeted values. A custom pH balanced perfusion specific crystalloid solution had been used in the past. This simple crystalloid solution was beneficial but another problem that was not solved by the customized crystalloid solution was the unmet need for removing fluid quickly via a hemofilter. As mentioned before, the electrolytes are a major focus during the surgeries, but one of the most important factors that is directly affected while the blood is “washed” is the hematocrit. As volume is added to the patient to dilute the electrolytes it also dilutes the total red cell volume. Acute changes like this can affect the oxygen carrying capacity of the patient, the vascular tone, and can impact end organ perfusion. Hemofiltration is a great tool but very slow relative to a rapid paced cardiac surgery. The limitation is that one cannot add a large bolus of fluid to a patient repeatedly because of its negative affects so the fluid must be parceled and removed, add fluid then remove fluid and add fluid then remove fluid. This is where the term “washing” the blood originates. The problem is that the excessive amount of volume takes a certain amount of time to remove before the process can continue. The inventors contemplated modifying or manipulating the hemofilter to increase its efficiency when used in conjunction with customized physiologic crystalloid solutions.

The theory of infusing by electrolyte counter current to the blood being shunted through the hemofilter would cause a diffusion of the electrolytes without the need of adding the volume into the patient's blood stream. As the blood passes through the hemofilter fibers and the optimized electrolyte solution moves around those fibers the electrolytes seek an equilibrium from the electrolyte gradient between the patient's blood going through the inside of the fibers of the hemofilter versus the electrolytes in the custom crystalloid flowing counter current on the outside of the fibers.

The device described herein is a small, light-weight, self-contained system that can be adapted to any cardiopulmonary bypass Heart/Lung machine, extracorporeal membrane oxygenation console, or stand-alone unit for restoration of undesirable banked blood.

### A. Extracorporeal Conditioning System

Device described herein can be operated in 1, 2, 3, 4 or more different modes: (1) “zero balanced” electrolyte transfer, (2) fluid removal, (3) a combination of both electrolyte transfer and fluid removal simultaneously, and (4) physiological restoration of banked blood. Buttons can be present to select modes or dictate pump speeds during the combo mode. This system can provide a more precise and efficient level of ultrafiltration, thus allowing the patient to maintain an optimum pH and electrolyte balance during heart surgery, during application of ventricular assist devices (VAD's), as well as during extracorporeal membrane oxygenation (ECMO) runs.

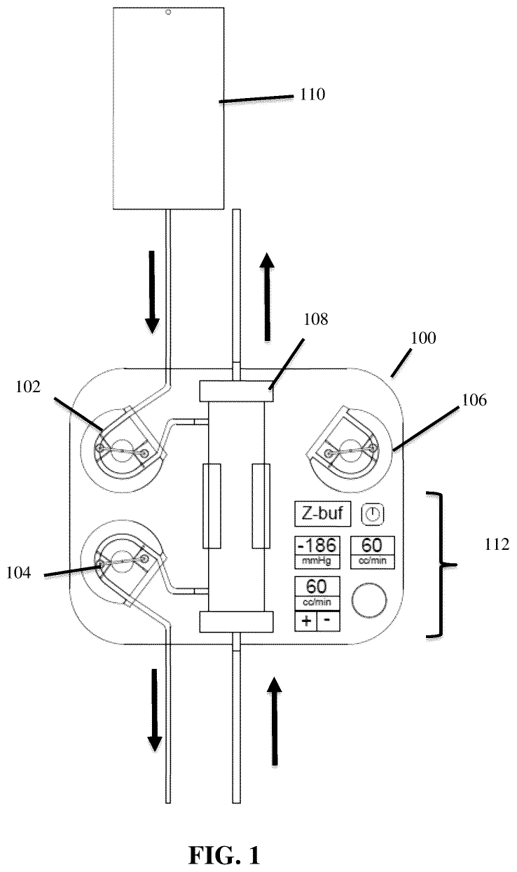

Referring to FIG. 1, the system is comprised of console 100 with dual roller pumps 102 and 104 to be used in conjunction with preassembled sterile disposable, hemofilter 108 as well as an optimum pH and electrolyte balanced cardiovascular solution 110. Multiple displays 112 on the console show each pumps flow rate (ml/min) and the pressure (mmHg) on the negative side of the outlet pump.

Hemofilters are used in many cardiovascular cases. Blood is usually shunted through the hemofilter which contains hundreds of semipermeable microporous fibers and then reenters the patients cardiopulmonary support circuit. The pores are small enough to allow only small molecules, like aqueous solutions, electrolytes or anything else that is smaller than 50,000 Daltons. If molecules are greater than 50,000, like plasma proteins, blood cells, and most other blood components, they will not pass through the pores and will remain in the circuit and patient circulation. As a negative pressure is applied to the hemofilter, the aqueous fluid is removed which essentially concentrates the contents of the blood.

Continuing reference to FIG. 1, the two roller pumps (e.g., 102 and 104 of FIG. 1), designated as the inlet (102) and outlet (104) pumps can perform synchronously or independently from each other. For different situations different modes have been incorporated, for example Z-buf (1), Concentration (2), Combo (3) and Restoration (4). The different modes can be utilized during the following examples. (i) During heart surgery, it is not uncommon for the patient's pH and electrolytes to become out of balance due to multiple outside factors such as normal saline (pH 5.0) injection from the surgical table or doses of high potassium cardioplegia solution (K>7). Traditional treatment of these issues would be the administration of sodium bicarbonate for pH correction and the addition of 0.9% NaCl to the patient's bypass circuit to reduce an elevated potassium level by diluting it. The diluted volume is then removed via ultrafiltration. This method is time consuming and inefficient and other electrolytes could subsequently become imbalanced such as chloride. With the device/system described herein the top pump or inlet pump 102 pulls the sterile pH/electrolyte balanced cardiovascular solution from bag 110 and pumps it into hemofilter 108. This solution moves counter current outside of the fibers, as the blood pathway moves inside the hemofilter fibers. Outlet pump 104 draws fluid from within the hemofilter and can expel it to a proper waste container. With safety in mind, a pressure monitor is attached on the negative side of outlet pump 104 from hemofilter 108, in order to assure that a negative pressure is maintained. Safe guards can be provided or built in to stop both the pumps if the pressure becomes positive or if it becomes too negative. Another safety feature that can be incorporated is that the inlet pump that pushes fluid into the hemofilter can be regulated to never to run at a higher flow rate than the outlet pump which pulls fluid from the system. As the pH/electrolyte balanced cardiovascular solution runs through the hemofilter, the electrolytes within the blood shift to establish an equilibrium between the solution and the blood. If undesirable or even dangerous electrolyte levels are present this device can be employed to rapidly, yet safely, reduce and balance the pH and electrolyte levels to their normal physiological state in a very short amount of time. When inlet 102 and outlet 104 pumps move at the same speed there is a net zero balance of fluid between the patient and the electrolyte transfer system.

In the event that only fluid removal is necessary from the patient bypass circuit, the system has a mode that will remove fluid more efficiently than the traditional ultrafiltration method of vacuum only. This mode will require that the inlet pump does not rotate and the outlet pump responds to the users designated removal rate. Also, included in this system is a mode which is a combination of the counter current technique with additional fluid removal. This combo mode can be used in the event that the patient's electrolytes require to be normalized, but fluid also needs to be removed. For example electrolyte imbalances can occur when the hematocrit needs to be elevated. This can occur because even “fresh” packed red blood cells typically have an elevated potassium and pH imbalance. With the combo mode engaged one can increase the hematocrit and restore the optimum physiologic state of the blood in a matter of minutes.

In certain embodiments the console is electrically powered, small, light-weight and water resistant. A bracket can be provided, preferably on the back side, so the console can easily be attached to any brand of cardiopulmonary bypass machines or ECMO consoles. The accompanying sterile disposable can come fully assembled and easily pops onto the device.

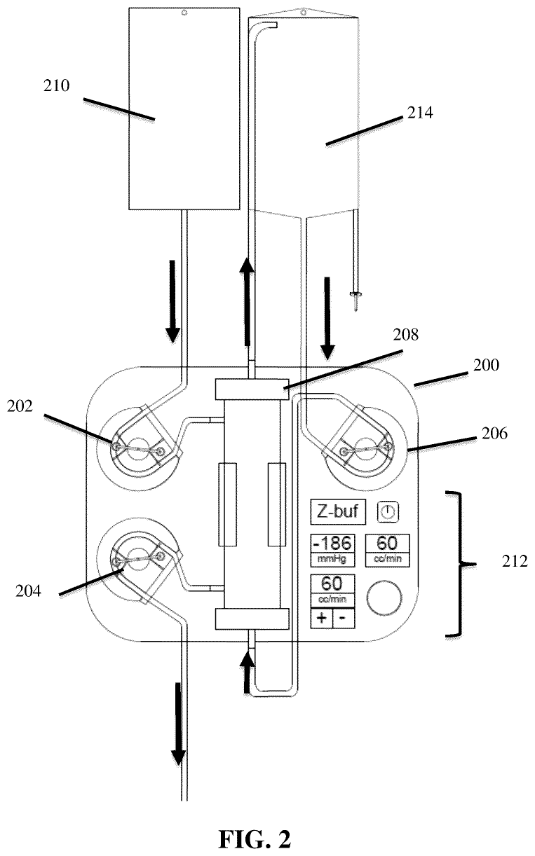

The sterile disposable can be used as an additional component to a cardiovascular bypass system, the primary use. The device has a secondary use which can be implemented as a stand-alone unit. With reference to FIG. 2, the sterile disposable is functionally different and can be used for the restoration of banked blood. The two different systems can employ custom pH/electrolyte balanced cardiovascular solutions. For the primary use the pH/electrolyte balanced cardiovascular solution can be broken down into two types. Solution A will be composed of a physiologic pH balanced crystalloid that includes sodium, calcium, chloride and glucose within their normal ranges. Solution B will contain the same components but will also incorporate potassium in a normal physiologic range. The secondary use disposable will also include a pH balance crystalloid solution similar to solution B but it will not contain calcium. The calcium will interfere with the anticoagulant, citrate, of the banked blood causing it to clot.

The system illustrated in FIG. 2 includes console 200 having inlet pump 202, outlet pump 204 in communication with solution bag 210 and hemofilter 208. Console 200 has display 212. The device is also configured to be in communication with blood source 214 which is in communication with hemofilter 208. The blood is counter flow circulated relative to the solution provided by solution bag 210 by pump 206.

### B. Methods of Regulating Electrolyte Levels

The devices, systems, and methods described herein could be used safely on any cardiopulmonary bypass circuit, ECMO, or temporary ventricular assist device. This device could be used to normalize a cardiopulmonary circuit before it is used on a patient. As a unit of blood ages, the cell wall of the red blood cell begins to deteriorate and can eventually rupture. The patient will be exposed to the noxious contents of the ruptured cell if they are transfused with this blood. The secondary use of the device/system will help normalize the electrolytes, primarily potassium and glucose of banked blood. The speed, efficiency, and portability of the device in tandem with the custom crystalloid solutions, and sterile disposables make it a unique system that can be utilized in various hospital settings.

The traditional method for fluid removal has been to simply remove fluid with a hemofilter from the cardiopulmonary bypass circuit. The device/system described herein allows for fluid removal as well as a counter current fluid transfer to quickly manipulate the patient's electrolytes or return banked blood to a physiologic state. The pH/electrolyte balanced solutions in conjunction with the counter current flow is distinct from the current industry wide practice.

With the proper pH/electrolyte balanced cardiovascular solution the inventors have shown proof of concept. The unexpected results encountered was extreme efficiency of the system.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Maintaining Physiological Status While on Cardiopulmonary Bypass Utilizing Electrolyte Transfer and Custom Perfusion Ph Balanced Solutions

A representative system was tested on a circuit of expired blood with adverse pH/electrolyte levels. The device was used with a pH/electrolyte balanced cardiovascular solution and the pH/electrolyte levels were normalized within minutes of starting the mechanism. In one experiment the potassium was lowered from 8.7 to 3.9 in approximately 6 minutes. This is a significant achievement when compared to the traditional method.

### A. Materials and Methods

Studies were performed to recreate a clinical setting and compare an embodiment of the current invention to traditional methodologies. The improved counter current system described herein and custom pH balanced crystalloid solution is compared to the industry wide practice of the use of traditional crystalloids with traditional hemofiltration, which would serve as the control group. After a cardiac surgery takes places there is occasionally a portion of the patient's blood left in the cardiopulmonary bypass machine that will be discarded once the patient's surgery is complete and the patient has left the operating room. Studies utilized this discarded human blood. The electrolyte levels are manipulated to mimic the undesirable levels one may experience on bypass. Once the mimic conditions were established the inventors would attempt to compare the two techniques. Also, the blood bank at times has expired blood and fresh frozen plasma, which can be used to prime the circuits to simulate situations of undesirable levels of the electrolytes while on cardiopulmonary bypass.

One disclaimer is that the inventors chose to use their own wash solution noted as “Wash A” because it is the inventor's clinical experience that this is an optimal crystalloid when compared to normal saline. Wash A has a sodium level of 138 mmol/L and balance pH, where 0.9% NaCl has a sodium of 154 and a pH of 5.0. Over a 12 minute span 305 mL of packed red blood cells were given to increase the hematocrit and 600 mL of Wash A was added to lower the potassium level. A reminder is that this large amount of volume added to a circuit takes a certain amount of time to reach its original volume when using a hemoconcentrator. In this 12 minutes a 14% drop in the potassium was experienced, the wash was continued for an additional 18 minutes with 600 mL added and 57% drop was recorded. At this point the inventors were 30 minutes into the traditional method with 1200 mL of wash used. It is noteworthy to observe the other electrolytes during this process and their changes. Sodium bicarbonate and calcium were added and then an additional 13 minutes of Wash B was used. Wash B is similar to Wash A, but it contains a physiologic level of potassium. The end result was a circuit blood gas that resembled the patient's baseline, the problem is that it took 51 minutes and 1500 mL of washing to achieve this.

### B. Results

In the traditional method, table 1, an actual patient baseline gas is provided in column 1, the first gas evaluated on the circuit is in column 2. It is noted that the potassium has been artificially elevated. One disclaimer is that the inventors chose to use their own wash solution noted as “Wash A” because it is the inventor's clinical experience that this is an optimal crystalloid when compared to normal saline. Wash A has a sodium level of 138 mmol/L and balance pH, where 0.9% NaCl has a sodium of 154 and a pH of 5.0. Over a 12 minute span 305 mL of packed red blood cells were given to increase the hematocrit and 600 mL of Wash A was added to lower the potassium level. A reminder is that this large amount of volume added to a circuit takes a certain amount of time to reach its original volume when using a hemoconcentrator. In this 12 minutes a 14% drop in the potassium was experienced, the wash was continued for an additional 18 minutes with 600 mL added and 57% drop was recorded. At this point the inventors were 30 minutes into the traditional method with 1200 mL of wash used. It is noteworthy to observe the other electrolytes during this process and their changes. Sodium bicarbonate and calcium were added and then an additional 13 minutes of Wash B was used. Wash B is similar to Wash A, but it contains a physiologic level of potassium. The end result was a circuit blood gas that resembled the patient's baseline, the problem is that it took 51 minutes and 1500 mL of washing to achieve this.

TABLE 1
Traditional Method

| | | | | | 8 minutes | |
|---|---|---|---|---|---|---|
| | | | 12 minutes | | Added 6 | 13 minutes |
| | | | 305 mL | | mEq | Sweep |
| | | | PRBC and | | NaHCO₃ | Adjustment |
| | | | 600 mL | 18 minutes | and | and 300 mL |
| | | | Wash A | 600 mL | 250 mg | Wash |
| Traditional Method | Patient Gas | Circuit | Added | Wash A | CaCl | B |
| pH | 7.42 | 7.18 | 7.19 | 7.35 | 7.48 | 7.41 |
| Sodium (mmol/L) | 137 | 137 | 138 | 137 | 144 | 142 |
| Potassium (mmol/L) | 3.9 | 11.4 | 9.8 | 4.2 | 3.9 | 3.8 |
| Calcium (mmol/L) | 1.21 | 1.25 | 0.63 | 0.42 | 1.91 | 1.05 |
| Chloride (mmol/L) | 108 | 106 | 109 | 111 | 116 | 105 |
| Glucose (mg/dL) | 116 | 313 | 249 | 128 | 119 | 66 |
| Base Excess (mmol/L) | −3.1 | −12 | −13.8 | −5.2 | 3.9 | 1.2 |
| Bicarb (mmol/L) | 20.8 | 15.3 | 13.4 | 19.9 | 27.6 | 26 |
| Hematocrit (%) | 29 | 17 | 32 | 27 | 29 | 29 |
| Lactate | 1.0 | 8.0 | 7.9 | 4.6 | 4.7 | 3.0 |

51 minutes and 1500 mL of Wash A added

In the rapid electrolyte transfer experiment (table 2) the countercurrent system was set up on the circuit and no fluid was added into the mock patient's circulation. The efficiency of the system was immediately apparent. Wash A was used and within 3 minutes the inventors had achieved a 26% decrease in the potassium level while maintaining the pH and hematocrit. The process was continued for 3 additional minutes and an additional 39% decrease in the potassium was achieved which ultimately restored it to a physiologic level. In this experiment the glucose and lactate were reduced yet the pH, sodium and hematocrit were maintained. An overall 55% reduction is potassium was accomplished in 6 minutes.

TABLE 2
Rapid ETS method

| | | 3 Minutes | 3 Minutes |
|---|---|---|---|
| | Simulated | 300 mL of | 300 mL |
| Rapid ETS Method | circuit Gas | Wash A | Wash A |
| pH | 7.33 | 7.35 | 7.38 |
| Sodium (mmol/L) | 130 | 134 | 137 |
| Potassium (mmol/L) | 8.7 | 6.4 | 3.9 |
| Calcium (mmol/L) | 1.05 | 0.9 | 0.69 |
| Chloride (mmol/L) | 100 | 101 | 102 |
| Glucose (mg/dL) | 249 | 208 | 153 |
| Base Excess (mmol/L) | −3.1 | −1.4 | 0.6 |
| Bicarb (mmol/L) | 22.7 | 24.3 | 26 |
| Hematocrit (%) | 30 | 31 | 31 |
| Lactate | 2.3 | 2.0 | 1.5 |

6 minutes and 600 mL of zero balance

An additional method was conceived that could use a slow and continuous method to maintain the physiology versus making quick drops in the potassium. Wash A and B were both used in a countercurrent method. The countercurrent system was run at a much slower pace to measure its effects. 60 mL was used every 3 minutes versus the 300 mL in the previous electrolyte transfer experiment and the results were still favorable. The initial 3 minutes saw a 22% drop in potassium, then 12% on the next 3, 16% and then 11.6%. This brought potassium to a normal range and wash B was used.

The results were promising. Never before have the inventors been able to influence targeted electrolytes without significantly impacting others. It's almost as though one problem is fixed while another problem is created. The speed and efficiency is something that was unexpected when the experiment was initiated. Based on these results an Electrolyte transfer system used in conjunction with an optimized perfusion specific crystalloid solution is much better than the industry wide standard.

TABLE 3
Continuous ETS Method

| | | 3 minutes | 3 minutes | 3 minutes | 3 minutes | 3 minutes | 2 minutes |
|---|---|---|---|---|---|---|---|
| Continuous | Simulated | 60 mL | 60 mL | 60 mL | 60 mL | 60 mL | 40 mL |
| ETS Method | Patient Gas | WashA | WashA | WashA | WashB | WashB | WashB |
| pH | 7.49 | 7.46 | 7.46 | 7.46 | 7.47 | 7.45 | 7.43 |
| Sodium | 135 | 137 | 138 | 139 | 140 | 140 | 140 |
| (mmol/L) | | | | | | | |
| Potassium | 9.0 | 7.0 | 6.1 | 5.1 | 4.5 | 4.1 | 4.0 |
| (mmol/L) | | | | | | | |
| Calcium | 1.33 | 1.31 | 1.30 | 1.29 | 1.29 | 1.28 | 1.25 |
| (mmol/L) | | | | | | | |
| Chloride | 98 | 97 | 98 | 98 | 99 | 97 | 95 |
| (mmol/L) | | | | | | | |
| Glucose | 216 | 188 | 176 | 165 | 159 | 150 | 137 |
| (mg/dL) | | | | | | | |
| Base Excess | 7.2 | 4.9 | 4.9 | 4.9 | 5.6 | 4.1 | 2.6 |
| (mmol/L) | | | | | | | |
| Bicarb | 31.2 | 29.2 | 29.2 | 29.2 | 29.8 | 28.5 | 27.2 |
| (mmol/L) | | | | | | | |
| Hematocrit | 38 | 38 | 37 | 36 | 36 | 38 | 37 |
| (%) | | | | | | | |
| Lactate | 3.9 | 3.3 | 3.1 | 2.8 | 2.7 | 2.5 | 2.2 |

20 minutes and 340 mL of zero balance

1-50. (canceled)

51. A system for adjusting a concentration of at least one electrolyte in a patient's blood during extracorporeal circulation, comprising:
a reusable console including a first pump and a second pump;
a sterile disposable module including a hemofilter having a first filter chamber configured to conduct blood flow and a second filter chamber, the first filter chamber separated from the second filter chamber by a semipermeable barrier, the first filter chamber coupled to blood flow tubing at a blood input port and a blood output port, respectively, the blood flow tubing configured for connection to an extracorporeal circuit;
a wash solution reservoir in fluid communication with the second filter chamber, the second filter chamber including a wash solution inlet port and an effluent outlet port, the first pump configured to deliver a wash solution from the wash solution reservoir to the second filter chamber through the wash solution inlet port, the second pump configured to remove effluent from the second chamber;
an electrolyte sensor in communication with the first filter chamber and configured to collect electrolyte concentration data in the first filter chamber; and
a processor configured to receive the electrolyte concentration data from the electrolyte sensor and control rates of the first and second pumps to control a rate of flow of a wash solution from the wash solution reservoir through the second filter chamber based on electrolyte concentration data.

52. The system of claim 51, wherein the wash solution flows in a first direction through the second filter chamber and the patient's blood flows in a second direction through the first filter chamber, the first direction being opposite the second direction.

53. The system of claim 51, wherein the sterile disposable module is configured for single-use and is configured for easy attachment to the extracorporeal circuit.

54. The system of claim 51, wherein the semipermeable barrier includes pores sized to permit diffusion of electrolytes while preventing plasma proteins, blood cells and other blood components from passing through the pores.

55. The system of claim 51, wherein the electrolyte sensor is configured to detect the electrolyte concentration of at least one of potassium, sodium, calcium, chloride, bicarbonate, glucose and hydrogen ion in the patient's blood.

56. The system of claim 51, wherein the processor is configured to cause a chance in speed of at least one of the first pump and the second pump based on the electrolyte concentration data.

57. The system of claim 51, wherein the processor is configured to maintain the electrolyte concentration data within a desired range.

58. The system of claim 57, wherein the desired range is comprised of one of a hydrogen ion pH of seven and thirty-five hundredths to seven and forty-five hundredths (7.35-7.45), a sodium level of one hundred thirty-five to one hundred forty-five millimoles per liter (135-145 mM), a potassium level of three and one-half to four and one-half millimoles per liter (3.5-4.5 mM), a calcium level of eight tenths to one and two tenths millimoles per liter (0.8-1.2 mM), a chloride level is ninety five to one hundred fifteen millimoles per liter (95-115 mM), a glucose level is eighty to one hundred twenty milligrams per deciliter (80-120 mg/dL), a bicarbonate level is twenty-two to twenty-eight millimoles per liter (22-28 mM) and a lactate level of zero to two millimoles per liter (0-2 mM).

59. The system of claim 51, further comprising:
a pressure sensor in fluid communication with the processor, the processor configured to adjust a rate of the first pump and the second pump based on a pressure differential between the first filter chamber and the second filter chamber.

60. The system of claim 51, wherein the processor causes a change in speed of at least one of the first pump and the second pump based on the electrolyte concentration data.

61. The system of claim 51, wherein the processor is configured to direct the first and second pumps to move at the same speed in a zero balanced electrolyte transfer mode.

62. The system of claim 51, wherein the system operates in at least one of (a) a zero-balance electrolyte transfer mode, (b) a fluid removal mode and (c) a combined electrolyte transfer and fluid removal mode.

63. The system of claim 51, wherein the wash solution comprises a customized crystalloid solution.

64. The system of claim 51, further comprising:
a second wash solution configured to flow through the second filter chamber after flow of the wash solution through the second filter chamber is complete.

65. The system of claim 51, wherein the hemofilter is comprised of a preassembled sterile disposable hemofilter.

66. The system of claim 51, further comprising:
a bracket mounted to a back side of the console, the bracket configured for attachment to at least one of a cardiopulmonary bypass machine, a temporary ventricular assist device and an extracorporeal membrane oxygenation (“ECMO”) machine.

67. The system of claim 51, wherein the console is easily mountable to or pops onto at least one of a cardiopulmonary bypass machine and an extracorporeal membrane oxygenation machine.

68. The system of claim 51, wherein a rate of flow of the patient's blood through the first filter chamber is within a flow range of twenty to two hundred milliliters per minute (20-200 ml/min).

69. A method for adjusting a concentration of an electrolyte in a patient's blood during extracorporeal circulation with a system having a reusable console with a first pump, a second pump and a sterile disposable module with a hemofilter, the method comprising:
connecting the console to an extracorporeal circuit using blood flow tubing;
flowing the patient's blood through a first filter chamber of the hemofilter, the blood flow tubing connected to a blood input port and a blood output port of the hemofilter, the blood input port and the blood output port being in fluid communication with the first filter chamber;
flowing a wash solution through a second filter chamber of the hemofilter in a direction counter current to the patient's blood;
measuring at least one of a pressure using a pressure sensor and electrolyte concentration data using an electrolyte sensor;
receiving the at least one of the pressure and the electrolyte concentration data at a processor; and
adjusting a rate of at least one of the first and second pumps with the processor based on the at least one of the pressure and the electrolyte concentration data.

70. The method of claim 69, wherein the electrolyte sensor is configured to collect the electrolyte concentration data, which includes at least one of potassium, sodium and bicarbonate, the processor configured to adjust the rate of the at least one of the first and second pumps to maintain physiologic conditions of the patient's blood.

71. The method of claim 69, further comprising:
connecting the console to an extracorporeal membrane oxygenation machine.

72. The method of claim 69, further comprising:
displaying a first flow rate of the first pump, a second flow rate of the second pump and a pressure on a negative side of the second pump on a display on the console.
